SEQUENCE LISTING (6 sequences)

SEQ ID NO: 1 - DNA, 30 bp, canine parvovirus
aaacaggaattaactatactaatatattta

SEQ ID NO: 2 - DNA, 23 bp, canine parvovirus
aaatttgaccatttggataaact

SEQ ID NO: 3 - DNA, 30 bp, canine parvovirus
tggtcctttaactgcattaaataatgtacc

SEQ ID NO: 4 - DNA, 27 bp, canine distemper virus
agctagtttcatcttaactatcaaatt

SEQ ID NO: 5 - DNA, 23 bp, canine distemper virus
ttaactctccagaaaactcatgc

SEQ ID NO: 6 - DNA, 30 bp, canine distemper virus
acccaagagccggatacatagtttcaatgc

(19) United States
(12) Patent Application Publication
(10) Pub. No.: US 2021/0363514 A1
(43) Pub. Date: Nov. 25, 2021

(54) SYRINGE-TYPE NUCLEIC ACID EXTRACTION APPARATUS, NUCLEIC ACID EXTRACTION KIT AND NUCLEIC ACID EXTRACTION METHOD THEREWITH

(71) Applicant: Wizbiosolutions lnc., Seongnam-si (KR)

(72) Inventors: Hyun Young LEE, Seongnam-si (KR); Jin Seok YU, Seongnam-si (KR)

(21) Appl. No.: 17/323,604

(22) Filed: May 18, 2021

(30) Foreign Application Priority Data
May 22, 2020 (KR) 10-2020-0061312

(51) Int. Cl. C12N 15/10; B01L 3/00

(52) U.S. Cl. CPC C12N 15/1017; B01L 3/523; B01L 2400/0478; B01L 2300/047; B01L 2300/0672; B01L 3/502

(57) ABSTRACT

Provided is a syringe-type nucleic acid extraction apparatus, a nucleic acid extraction kit capable of simply and quickly separating nucleic acid without the aid of electronic devices or other mechanical devices, and a method for extracting the nucleic acid. According to the present disclosure, the nucleic acid extraction apparatus includes a syringe contacting an upper surface of the nucleic acid purification filter and including a cylinder and a piston; a tip having an upper surface in contact with a lower surface of the nucleic acid purification filter and a lower side through which washing liquid or a nucleic acid elution buffer moves; and a coupler configured to connect a lower end of the syringe to the upper end of the tip and into which the nucleic acid purification filter is inserted.

### CROSS-REFERENCE TO RELATED APPLICATION

This application claims priority to and the benefit of Korean Patent Application No 10-2020-0061312, filed on May 22, 2020, the disclosure of which is incorporated herein by reference in its entirety.

### BACKGROUND

### 1. Field of the Disclosure

The present disclosure relates to a syringe-type nucleic acid extraction apparatus, a nucleic acid extraction kit, and a nucleic acid extraction method using the same, and more particularly, to a syringe-type nucleic acid extraction apparatus and a nucleic acid extraction kit capable of purifying nucleic acid simply and quickly without aid of electronic devices or other mechanical devices, and a nucleic acid extraction method using the same.

### 2. Description of Related Art

In recent years, importance of research on analysis and interpretation of genetic information such as a genome or DNA, transcriptome, and RNA is increasing in the fields of disease diagnosis, treatment strategy establishment, treatment monitoring, and the like.

Separation of nucleic acid (e.g., DNA and RNA) from a biological specimen is the most important step in biochemical research and diagnostic processes. If the nucleic acid as a genetic material is not separated from the specimen, the next steps such as gene detection, gene cloning, gene sequencing, gene amplification, and cDNA synthesis may not be performed. An effective and reproducible separation method is needed to differentiate the DNA or the RNA from a mixture of cells.

A method for extracting the nucleic acid in related art include a method of separating nucleic acids absorbed on a magnet using magnetic beads, a method of introducing air into a column and pushing the solution out to elute the solution with the column, and a method of eluting a solution by centrifuging the column.

The nucleic acid extraction equipment using the above method has a relatively large size to process a large amount of specimens; however, time for processing the specimen may take too long. In addition, contamination due to each specimen may occur in the process of processing a large number of specimens, thereby reducing processing efficiency and causing user inconvenience.

The most widely used nucleic acid extraction method is a nucleic acid extraction method using the principle of adsorption chromatography. The method enables adsorption by placing a silica membrane on an extraction kit or coating silica on the magnetic bead. In the environment of extraction solution having low pH, high ionic strength, and with chaotropic salts, the nucleic acids having negative (−) polarity is adsorbed on a silica surface, the adsorbed nucleic acid is washed, and subsequently, and the nucleic acid is eluted with an elution buffer having high pH and low ionic strength.

However, when the chaotropic salts are included in the purified nucleic acid, the chaotropic salts act as a reaction inhibiting substance in a polymerase chain reaction (PCR) reaction to finally use the nucleic acid. For this reason, the nucleic acid has to undergo several washing processes during the purification of the nucleic acid, thereby making a cumbersome process.

### SUMMARY OF THE DISCLOSURE

The present disclosure provides a syringe-type nucleic acid extraction apparatus and a nucleic acid extraction kit capable of simply and quickly purifying nucleic acid without the aid of electronic devices or other mechanical devices, and a nucleic acid extraction method using the same.

The problem to be solved by the present disclosure is not limited to the problem(s) mentioned above, and other problem(s) not mentioned above will be clearly understood by those skilled in the art from the following description.

The present disclosure provides a syringe-type nucleic acid extraction apparatus. The syringe-type nucleic acid extraction apparatus includes a nucleic acid purification filter; a syringe contacting an upper surface of the nucleic acid purification filter and including a cylinder and a piston; a tip having an upper surface contacting a lower surface of the nucleic acid purification filter and a lower side through which washing liquid or nucleic acid elution buffer moves; and a coupler configured to connect a lower end of the syringe to the upper end of the tip and into which the nucleic acid purification filter is inserted.

The coupler includes an outer wall protrusion at a lower end of the syringe and an inner wall groove at an upper end of the tip. The outer wall protrusion and the inner wall groove may be male/female coupled.

The coupler may further include a structure to support the nucleic acid purification filter at a lower side of the syringe and an upper side of the tip.

The structure to support the nucleic acid purification filter at the lower side of the syringe may have a wagon wheel shape and the structure to support the nucleic acid purification filter at the upper side of the tip may include a plurality of right-triangular protrusions from an inner wall of the upper side of the tip. The nucleic acid purification filter may include one selected from nanofibers, glass fibers, or silica membranes.

The nucleic acid purification filter may be surface-treated with a cationic polymer.

In addition, the present disclosure provides a nucleic acid extraction kit further including a cartridge to process by adding the specimen, in addition to the syringe-type nucleic acid extraction apparatus.

The cartridge includes six wells and each well may be filled with a buffer needed to purify the nucleic acid or may be empty for drying.

In addition, the present disclosure provides a method for extracting nucleic acid. The method for extracting the nucleic acid includes (a) preparing a specimen by dissolving the sample in a lysis solution; (b) immersing the tip of the syringe-type nucleic acid extraction apparatus in the specimen prepared at the (a) and vertically reciprocating the piston of the syringe to adsorb nucleic acid of the prepared specimen to the nucleic acid purification filter; (c) immersing the tip in the washing liquid and vertically reciprocating the piston of the syringe to wash the nucleic acid purification filter; (d) vertically reciprocating the piston of the syringe to dry the nucleic acid purification filter; and (e) immersing the tip in the nucleic acid elution buffer and vertically reciprocating the piston of the syringe to separate nucleic acid in the nucleic acid purification filter.

At the step (a), the specimen may be prepared by dissolving the sample containing the nucleic acid in a solution of a nonionic surfactant.

The sample containing the nucleic acid may be one selected from the group consisting of whole blood, plasma, serum, blood card, saliva, feces, tissue cells, and sputum.

The washing at the (c) may be performed in three steps.

The syringe-type nucleic acid extraction apparatus, the nucleic acid extraction kit, and the nucleic acid extraction method using the same according to the present disclosure may enable rapid extraction of the nucleic acid without several washing processes during the purification thereof.

In addition, the syringe-type nucleic acid extraction apparatus, the nucleic acid extraction kit, and the nucleic acid extraction method using the same according to the present disclosure may enable simple extraction of the nucleic acid without need for assistance from other electronic devices or mechanical devices.

In addition, the syringe-type nucleic acid extraction apparatus, the nucleic acid extraction kit, and the nucleic acid extraction method using the same may enable the rapid and easy extraction of the nucleic acid from the specimen by inserting a nucleic acid purification filter into the syringe-type nucleic acid extraction apparatus. The effects of the present disclosure are not limited to the above effects, and should be understood to include all effects that may be deduced from the configuration of the disclosure described in the detailed description or claims of the present disclosure.

### BRIEF DESCRIPTION OF THE DRAWINGS

FIG. 1 is a schematic view of a syringe-type nucleic acid extraction apparatus according to an embodiment of the present disclosure.

FIGS. 2A and 2B are a cross-sectional view of a syringe-type nucleic acid extraction apparatus according to an embodiment of the present disclosure.

FIG. 3A is a perspective view showing a nucleic acid extraction kit cartridge according to an embodiment of the present disclosure. FIG. 3B is a bottom view of a nucleic acid extraction kit cartridge. FIG. 3C is a front view showing a nucleic acid extraction kit cartridge.

FIG. 4 is a process flowchart of a method for extracting nucleic acid according to an embodiment of the present disclosure.

FIG. 5 shows a process of extracting a nucleic acid from a specimen using a method for extracting nucleic acid according to an embodiment of the present disclosure.

FIG. 6 is a picture showing a result of amplifying a canine parvo virus DNA sample extracted according to an embodiment of the present disclosure and a control group by real-time polymerase chain reaction (PCR).

FIG. 7 is a picture showing a result of amplifying a canine distemper virus RNA sample extracted according to an embodiment of the present disclosure and a control group by Real-time RT-PCR

### DETAILED DESCRIPTION OF EXEMPLARY IMPLEMENTATIONS

In the following description, it should be noted that only parts necessary to understand embodiments of the present disclosure will be described, and descriptions of parts other than the above parts will be omitted without a scope that does not obscure the gist of the present disclosure.

Terms or words used herein and in claims, which are described below, should not be construed as being limited to a general or dictionary meaning, but should be construed as a meaning and a concept that conform to the technical idea of the present disclosure based on the principle that the inventor can properly define the concept of terms to describe his or her disclosure in the best way. The embodiments set for herein and the configurations shown in the drawings are merely preferred embodiments of the present disclosure and do not entirely represent the technical idea of the present disclosure. Therefore, it should be understood that various equivalents and modifications to replace them at the time of filing of this application can be made.

Hereinafter, the present disclosure will be described in detail with reference to the drawings.

Syringe-Type Nucleic Acid Extraction Apparatus

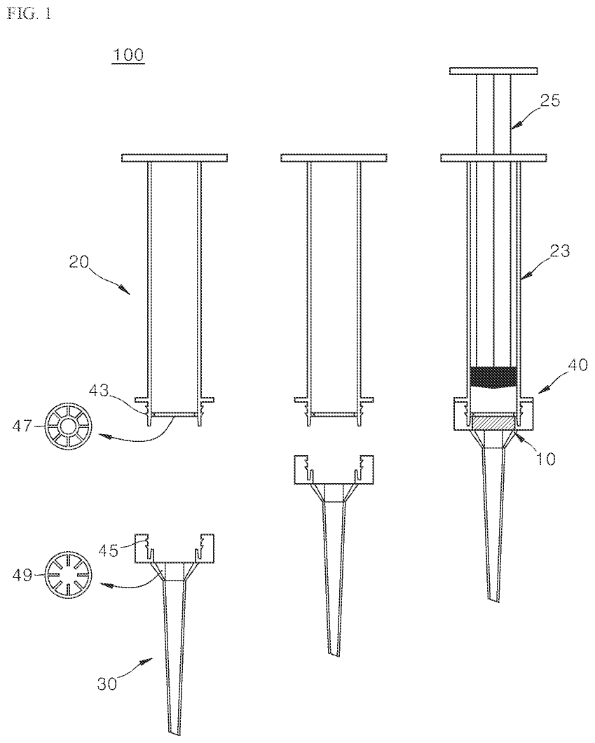

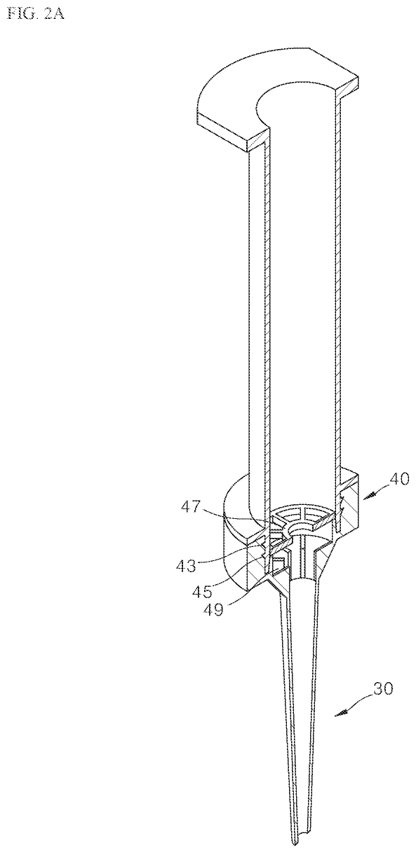

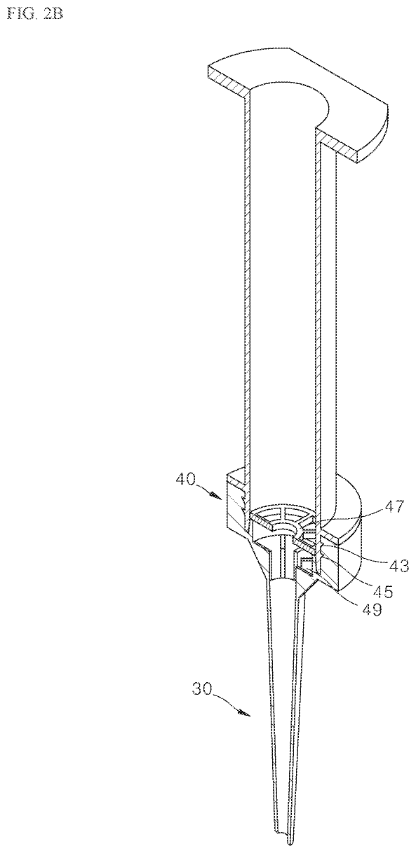

FIG. 1 is a schematic view of a syringe-type nucleic acid extraction apparatus according to an embodiment of the present disclosure. FIGS. 2A and 2B are a cross-sectional view of a syringe-type nucleic acid extraction apparatus according to an embodiment of the present disclosure Referring to FIG. 1, the present disclosure provides a syringe-type nucleic acid extraction apparatus including a nucleic acid purification filter 10, a syringe 20, a tip 30, and a coupler 40.

The syringe 20 contacts an upper surface of the nucleic acid purification filter 10 and includes a cylinder 23 and a piston 25. An upper surface of the tip 30 contacts a lower surface of the nucleic acid purification filter and prepared specimen, washing liquid, or nucleic acid elution buffer move through a lower side thereof.

The coupler 40 connects a lower end of the syringe to a upper end of the tip and the nucleic acid purification filter 10 is inserted into the coupler 40.

Referring to FIGS. 1, 2A and 2B, the nucleic acid purification filter 10 may have a disk shape, but the technical idea of the present disclosure is not limited thereto. That is, the nucleic acid purification filter may have various shapes, for example, a polygonal shape.

The nucleic acid purification filter may include one selected from nanofibers, glass fibers, and silica membranes.

The nanofiber has a diameter of several hundreds of nanometers (nm) or less and contains a plurality of pores. Therefore, when the nanofiber is used as the nucleic acid extraction filter, dozens of nano-sized or larger particles may be separated, thereby obtaining excellent nucleic acid collecting ability.

A material prepared by electrospinning a polymer material mixed with at least one of PVdF (polyvinylidenefluoride), PMMA (polymethylmethacrylate), PAN (polyacrylonitrile), PU (poly-urethane), PES (polyethersulfone), PAA (polyamic acid), PVA (polyvinylachol), PEG (polyethyleneoxide), PLA (polylacticacid), PGA (polyglycolic acid), and PLAPGA-based polymer material may be used as the nanofiber, but the nanofiber is not limited thereto.

The type of glass fiber or silica membrane is not particularly limited, and a glass fiber or silica membrane commonly used in the art may be used.

The nucleic acid purification filter 10 may be surface-treated with a cationic polymer.

In order to prepare the nucleic acid purification filter 10 surface-treated with the cationic polymer, the nucleic acid purification filter is immersed in a solution dissolving the cationic polymer, is washed with distilled water, dried, and then transmitted with ultraviolet rays to physically attach the cationic polymer on a surface of the nanofiber.

The cationic polymer may use one selected from the group consisting of polyethylenimine (PEI), 3-aminopropyl trimethoxysilane (APTMS), and chitosan, but is not limited thereto.

In this case, it is advantageously to immerse the nucleic acid purification filter in the solution dissolving the cationic polymer for 3 to 10 hours.

The solvent capable of dissolving the cationic polymer may be an acidic solution such as a mixed solvent of trifluoroacetic acid (TFA) and dichloromethane, a mixed solvent of hexafluoroisopropanol and trifluoroacetic acid, but is not limited thereto.

A concentration of the solution dissolving the cationic polymer may be 1.0 to 3.0% (w/v), but is not limited thereto.

The nucleic acid purification filter may be dried at 60 to 80° C. for 5 to 7 hours. The dried nucleic acid purification filter may be transmitted with ultraviolet rays at a wavelength of 290 to 320 nm for 30 minutes to 1 hour to physically attach the cationic polymer onto the surface of the nanofiber.

When the cationic polymer is treated on the surface of the nucleic acid purification filter according to the present disclosure, the cationic polymer having a (+) charge is condensed with the nucleic acid having a (−) charge to easily collect and extract the nucleic acid contained in the blood.

The syringe 20 contacts an upper surface of the nucleic acid purification filter 10 and includes a cylinder 23 and a piston 25.

A prepared specimen, washing liquid, or nucleic acid elution buffer may be contained in the cylinder 23.

The piston 25 may be pushed and pulled based on a vertical reciprocating motion to generate a negative pressure and a positive pressure. In this case, the solution contained in the cylinder 23 may be injected into the nucleic acid purification filter 10 or a specimen may be suctioned from the nucleic acid purification filter 10 to the cylinder 23.

The tip 30 has an upper surface in contact with a lower surface of the nucleic acid purification filter, and a prepared specimen, washing liquid, or nucleic acid elution buffer may move through a lower side thereof.

The coupler 40 connects the lower surface of the syringe to the upper surface of the tip and is a portion into which the nucleic acid purification filter 10 is inserted.

The coupler 40 includes an outer wall protrusion 43 at a lower end of the syringe and an inner wall groove 45 at an upper end of the tip. The outer wall protrusion 43 and the inner wall groove 45 may be male-female coupled. The outer wall protrusion 43 at the lower end of the syringe and the inner wall groove 45 at the upper end of the tip have a saw blade shape when viewed from the side as shown in FIG. 1 and both ends may be connected with one touch by applying a pressure thereto and pushing them.

The nucleic acid purification filter 10 may be inserted into the coupler 40. In addition, two or three nucleic acid purification filters may be inserted into the coupler 40, and in this case, the nucleic acid purification filters may be stacked inside and inserted into the coupler. According to the present disclosure, a nucleic acid may be quickly collected and extracted using the two or three nucleic acid purification filters.

In addition, when the nucleic acid purification filters are stacked inside the coupler, a pre-filter may be added to a lowermost layer of the nucleic acid purification filter According to the present disclosure, when the pre-filter is stacked on the lowermost layer of the nucleic acid purification filter, the stacked structure of the nucleic acid purification filter may be stably supported and blood specimens may be effectively filtered.

The coupler 40 connects the lower end of the syringe to the upper end the tip. Based on the vertical reciprocation of the piston 25, the solution may be introduced through the tip and then may be introduced into the cylinder 23 of the syringe through the nucleic acid purification filter 10 disposed inside the coupler, and subsequently, the solution may be discharged via the tip 30 through the nucleic acid purification filter 10 disposed in the coupler. When the syringe 20 is connected to the tip 30 through the coupler 40, the syringe 20 is completely integrated with the coupler 40 to block flowing-out of solution contained at an inside thereof.

The coupler 40 may further include a structure to support the nucleic acid purification filter at a lower side of the syringe and an upper side of the tip.

The structure to support the nucleic acid purification filter at the lower side of the syringe and the upper side of the tip may more firmly fix the nucleic acid purification filter inserted into the coupler.

A nucleic acid purification filter support structure 47 at the lower side of the syringe may have a wagon wheel shape and a nucleic acid purification filter support structure 49 at the upper side of the tip may have a plurality of right triangular protrusions from the inner wall of the tip.

The nucleic acid purification filter support structure 47 disposed at the lower side of the syringe may support the nucleic acid purification filter 10 inserted into the coupler 40 and may hold to block movement of the nucleic acid purification filter inserted into the coupler to the syringe when the piston 25 of the syringe reciprocates.

The nucleic acid purification filter support structure 49 disposed at the upper side of the tip may support the nucleic acid purification filter inserted into the coupler 40. In addition, because the nucleic acid purification filter support structure 49 has an opening at a central portion thereof, a specimen suctioned into the tip when the piston 25 of the syringe reciprocates may smoothly move to the syringe and may minimize residual solution in the coupler when the specimen is moved from the syringe to the tip.

Nucleic Acid Extraction Kit

The present disclosure may provide a nucleic acid extraction kit further including a cartridge to process by adding a specimen in addition to the syringe-type nucleic acid extraction apparatus.

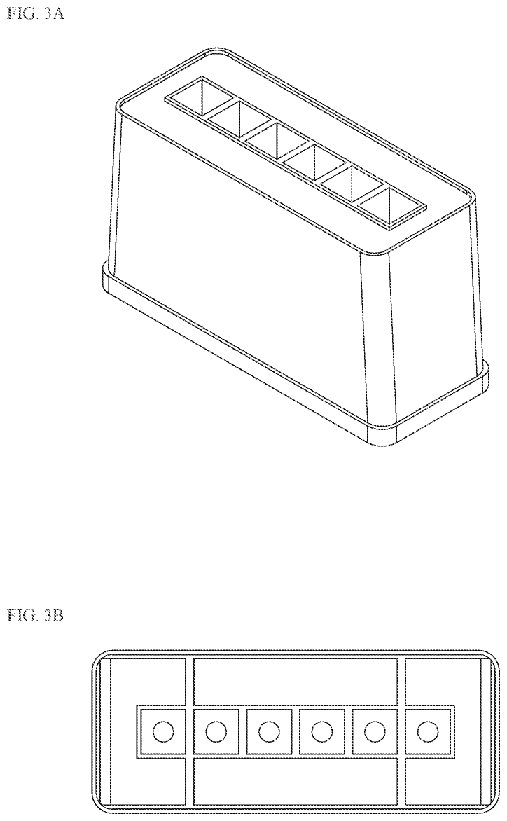

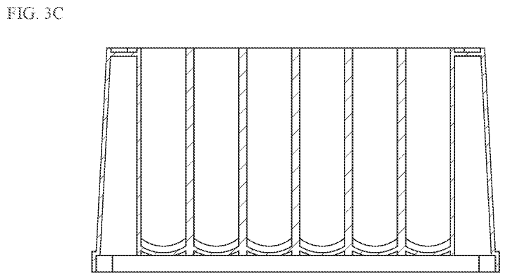

FIG. 3A is a perspective view showing a nucleic acid extraction kit cartridge according to an embodiment of the present disclosure. FIG. 3B is a bottom view showing a nucleic acid extraction kit cartridge FIG. 3C is a front view showing a nucleic acid extraction kit cartridge.

Referring to FIGS. 3A to 3C, the cartridge may include six wells, and each well may define a rectangular parallelepiped space.

A buffer to purify nucleic acid may be added to each of the wells or may be empty for drying.

More specifically, a nucleic acid lysis buffer may be added to well 1, a nucleic acid washing buffer may be added to wells 2 to 4, well 5 is empty for drying, and a nucleic acid separation buffer may be added to well 6.

Method for Extracting Nucleic Acid Using a Syringe-Type Nucleic Acid Extraction Apparatus

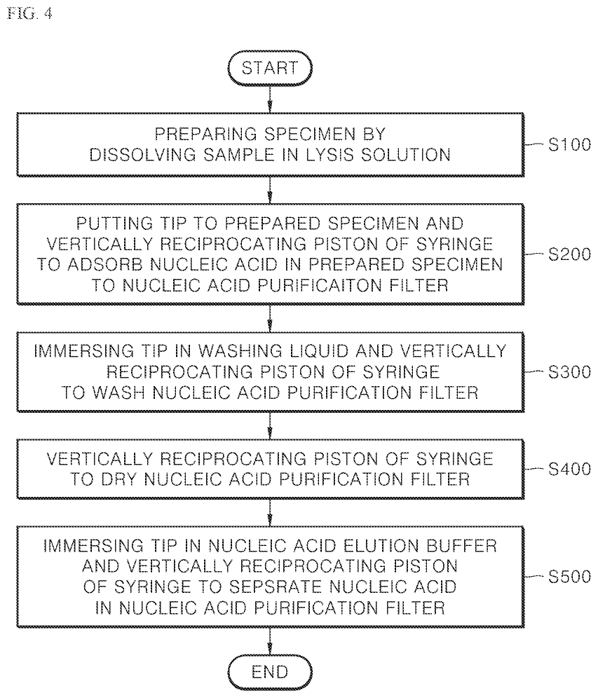

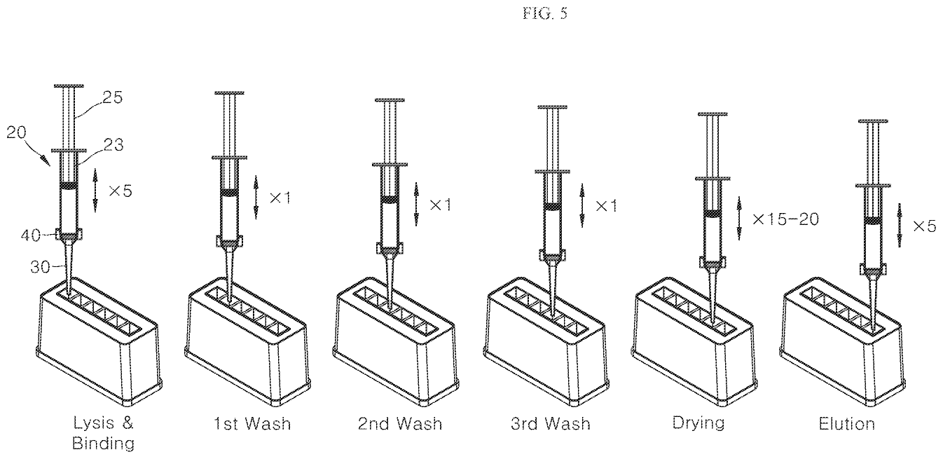

FIG. 4 is a process flowchart of a method for extracting nucleic acid according to an embodiment of the present disclosure. FIG. 5 is a picture showing a method for extracting nucleic acid using a syringe-type nucleic acid extraction apparatus and a nucleic acid extraction kit including a cartridge according to an embodiment of the present disclosure.

Referring to FIGS. 4 and 5, the present disclosure provides the method for extracting the nucleic acid. The method includes: (a) preparing a specimen by dissolving the sample in a lysis solution; (b) immersing a tip of a syringe-type nucleic acid extraction apparatus into the specimen prepared at the step (a) and vertically reciprocating a piston of a syringe to adsorb nucleic acid in the prepared specimen to a nucleic acid purification filter; (c) immersing the tip to washing liquid and vertically reciprocating the piston of the syringe to wash the nucleic acid purification filter; (d) vertically reciprocating the piston of the syringe to dry the nucleic acid purification filter, and (e) immersing the tip in a nucleic acid elution buffer and vertically reciprocating the piston of the syringe to separate nucleic acid in the nucleic acid purification filter.

The method for extracting the nucleic acid of the present disclosure may use the syringe-type nucleic acid extraction apparatus according to the present disclosure.

The method for extracting the nucleic acid of the present disclosure preferably uses the syringe-type nucleic acid extraction apparatus and the nucleic acid extraction kit including the cartridge according to the present disclosure, but the present disclosure is not limited thereto. The method for extracting nucleic acid of the present disclosure may use a container such as a tube commonly used in the art.

Hereinafter, the method for extracting the nucleic acid of the present disclosure is described in detail with reference to FIGS. 4 and 5.

In the method for extracting the nucleic acid according to an embodiment of the present disclosure, the step (a) is a step of preparing a specimen by dissolving the sample in a lysis solution (S100).

In the above step, the specimen may be prepared by dissolving the sample containing a nucleic acid in the lysis solution. The lysis solution may be a buffer solution or a nonionic surfactant solution in which a nonionic surfactant is dissolved in water or an aqueous buffer solution.

The sample containing the nucleic acid may be one selected from the group consisting of whole blood, plasma, serum, blood card, saliva, feces, tissue cells, and sputum, but is not limited thereto. That is, the sample containing the nucleic acid refers to all specimens that may contain nucleic acid and may include human and animal blood, plant body fluid, human and animal excrement, microbial culture solution, cell culture solution, virus culture solution, and the like.

The nucleic acid may include deoxyribonucleic acid (DNA) or ribonucleic acid (RNA).

The composition of the lysis solution may be appropriately adjusted according to DNA/RNA separation efficiency. The buffer may be selected from all biocompatible buffers, but is not limited thereto. The buffer having pH 7.0 to 8.5 may be used in consideration of biocompatibility.

The nonionic surfactant includes a hydrophilic portion and a hydrophobic portion in a molecule and has nonionic properties when being dissociated.

The nonionic surfactant may be one selected from the group consisting of Triton X-100, fatty alcohol ethoxylate (fatty acid ethoxylate (FAE)), polyethoxylated tallow amine (POEA), cocamidemonoethanolamine, cocamide diethanolamine, glycerol monostearate, glycerolmonolaurate, sorbitan monolaurate, sorbitan monostearate, sorbitan tristearate, Tween 20, Tween 40, Tween 60, Tween 80, decylglucoside, lauryl glucoside, octylglucoside, lauryldimethylamine oxide, phosphine oxide, but is not limited thereto.

The most effective concentration of the nonionic surfactant varies depending on types of surfactant, but is advantageously 1 to 20% relative to a total weight of the sample containing the nucleic acid. If the concentration of the nonionic surfactant is less than 1% or exceeds 20%, an extraction rate of the nucleic acid may be inhibited. % refers to w/v % or v/v % depending on types of the surfactant (e.g., sold or liquid).

In order to properly dissolve, the sample containing the nucleic acid may be treated with the lysis solution, and then reacted at 0 to 35° C. or 10 to 30° C. for 1 to 60 minutes, 3 to 30 minutes, or 5 to 20 minutes. However, the technical idea of the present disclosure is not limited thereto, and the temperatures and the time may be appropriately adjusted in consideration of types of samples, types or the concentration of the used lysis solution.

At the step (b), the tip of the syringe-type nucleic acid extraction apparatus is immersed to the specimen prepared at the step (a), and the piston of the syringe is reciprocated vertically to adsorb the nucleic acid in the prepared specimen to the nucleic acid purification filter (S200).

In the case of using the nucleic acid extraction kit according to the present disclosure, the lysis solution is added to well 1 of the cartridge, the sample is added to well 1 of the cartridge containing the lysis solution, and is mixed and reacted with the lysis solution. The tip of the nucleic acid extraction apparatus is put into well 1 of the cartridge containing the specimen prepared by mixing with the lysis solution and the piston of the syringe is vertically reciprocated 5 times to adsorb the nucleic acid in the prepared specimen to the nucleic acid purification filter of the nucleic acid extraction apparatus.

The step (c) is a step of immersing the tip in washing liquid and vertically reciprocating the piston of the syringe to wash the nucleic acid purification filter (S300).

The washing at the step (c) may be performed in three steps. In this case, the washing liquid commonly used in the art may be used as the washing liquid.

In the case of using the nucleic acid extraction kit according to the present disclosure, the tip of the nucleic acid extraction apparatus subjected to step (b) is placed in well 2 to which first washing liquid is added, and the piston of the syringe is reciprocated vertically once to clean the nucleic acid purification filter Subsequently, the tip of the nucleic acid extraction apparatus is placed in well 3 into which second washing liquid is added, and the piston of the syringe is reciprocated vertically once to wash the nucleic acid purification filter.

Finally, the tip of the nucleic acid extraction apparatus is placed in well 4 to which third washing liquid is added, and the piston of the syringe is reciprocated vertically once to wash the nucleic acid purification filter.

Step (d) is a step of drying the nucleic acid purification filter by reciprocating the piston of the syringe vertically (S400).

In the case of using the nucleic acid extraction kit according to the present disclosure, the tip of the nucleic acid extraction apparatus is put into empty well 5 of a cartridge and the piston of the syringe vertically reciprocates 15 to 20 times to dry the nucleic acid purification filter.

Step (e) is a step of immersing the tip into nucleic acid elution buffer and vertically reciprocating the piston of the syringe to separate the nucleic acid in the nucleic acid purification filter (S500).

In the case of using the nucleic acid extraction kit according to the present disclosure, the tip of the nucleic acid extraction apparatus is put into well 6 of a cartridge to which a nucleic acid elution buffer solution is added and the piston of the syringe vertically reciprocates 5 times to separate nucleic acid from a nucleic acid purification filter.

### Embodiment

Hereinafter, embodiments are described in detail to describe the present disclosure in detail. However, the embodiments according to the present disclosure can be modified in various other manners, and the scope of the present disclosure should not be construed as being limited to the embodiments described below.

Embodiments of the present disclosure are provided to more completely describe the present disclosure to a person having ordinary knowledge in the art.

<Preparation of Nucleic Acid Purification Filter and Syringe-Type Nucleic Acid Extraction Apparatus>

Nanofiber (Cellulose Mixed Esters, 25 mm, #CM045025, Macherey-Nagel) was prepared.

Nanofiber sheet No. 1 was treated with 1.25% to 2.5% (w/v) of PEI (Poly(ethyleneimine)). Nanofiber sheet No. 2 was treated with 1.25% to 2.5% (w/v) 3-aminopropyl trimethoxysilane Nanofiber sheet No. 3 was treated with 1.25% to 2.5% (w/v) of chitosan.

The nanofiber sheet was dried at 70° C. for 6 hours, and a surface of the dried nanofiber was treated with ultraviolet rays at a wavelength of 320 nm for 30 minutes using an ultraviolet irradiator. After the surface-treated nanofiber sheets Nos. 1 to 3 were cut to have a diameter of 10 mm, they were sequentially put into insertion positions of a nucleic acid purification filter disposed inside a coupler of the syringe-type nucleic acid extraction apparatus. Thereafter, a syringe and a tip of the syringe-type nucleic acid extraction apparatus were coupled and then adhered using ultrasonic waves.

<Preparation of Lysis Solution>

A lysis solution was prepared to be 100 mM of Tris-HCl (pH 8.0), 5% of TX-100, 2% of TW-20, 3% of Glycerol monolaurate, 0.5% of Sorbitan monostearate, and 0.25% of Lauryldimethylamine oxide.

<Preparation of Washing Liquid>

100 mM of Tris-HCl (pH 7.0), 5 mM of EDTA, and 1% of TX-100 were used as a first washing liquid, 50 mM of Tris-HCl (pH 7.0) was used as a second washing liquid, and 10 mM of Tris-HCl (pH 7.0) was used as a third washing liquid.

<Preparation of Elution Buffer>

10 mM of Tris-HCl (pH 8.0) and 0.1 mM of EDTA were used as a nucleic acid elution buffer.

<Preparation of Cartridge>

The prepared solution was dispensed into each well of a cartridge as follows. 1 ml of lysis solution was dispensed into well 1 of the cartridge, 1 ml of first washing liquid was dispensed into well 2, and 1 ml of second washing liquid was dispensed into well 3. 1 ml of third washing liquid was dispensed into well 4. Well 5 was left empty for drying. 0.5 ml of nucleic acid elution buffer was dispensed into well 6. An upper side of the cartridge after the liquid is dispersed is thermally bonded using a polyethylene (PE) film, thereby blocking leakage or mixing of solutions.

<Method for Extracting Nucleic Acid>

100 μl of blood as a sample was mixed with a lysis solution and was left at room temperature for 5 minutes. Subsequently, a syringe-type nucleic acid extraction apparatus was placed in well 1 containing the prepared specimen, and a piston of the syringe was reciprocated 5 times.

The syringe-type nucleic acid extraction apparatus was placed in well 2 and the piston of the syringe was reciprocated once.

The syringe-type nucleic acid extraction apparatus was placed in well 3 and the piston of the syringe was reciprocated once.

The syringe-type nucleic acid extracting apparatus was placed in well 4 and the piston of the syringe was reciprocated once.

The syringe-type nucleic acid extraction apparatus was placed in well 5 and the piston of the syringe was reciprocated 15 times.

The syringe-type nucleic acid extraction apparatus was placed in well 6 and the piston of the syringe was reciprocated 5 times to separate the nucleic acid from the nucleic acid purification filter.

### <Experimental Example 1> Extraction of Canine Parvo Virus DNA

<1-1> Purification of Virus DNA

100 μl of blood mixed with canine parvo virus (Central Vaccine Research Institute, CPV, 780916-LP, 105.0TCID50) was mixed with the lysis solution contained in well 1 of a cartridge, and then left at room temperature for 5 minutes to dissolve the blood A nucleic acid was separated and purified from a nucleic acid purification filter by reciprocating a piston of a syringe 5 times, reciprocating the piston once in well 2, reciprocating the piston once in well 3, reciprocating the piston once in well 4, reciprocating the piston 15 times in well 5, and finally reciprocating the piston 5 times in well 6. The finally eluted purified specimen was used as a template for a real-time polymerase chain reaction (PCR) reaction.

<1-2> PCR Execution

PCR was performed using virus DNA purified in Embodiment <1-1> as a template. As a control group for a method for extracting nucleic acid according to the present disclosure, a method of extracting CPV DNA with WizPure™ Viral DNA/RNA Mini Kit (Cat# W73050-100) of Wiz Bio Solution Inc. was used.

A forward primer 5′-AAACAGGAATTAACTATACTAATATATTTA-3′ (SEQ ID NO: 1), a reverse primer 5′-AAAITIGACCATTTGGATAAACT-3′ (SEQ ID NO: 2), and a probe primer 5′-FAM-TGGTCCTTTAACTGCATTAAATAATGTACC-BHQI-3′ (SEQ ID NO. 3) as primers used for real-time PCR were a capsid protein (VP2) gene of canine parvo virus.

Real-time PCR heats at 95° C. for 30 seconds using a WizDx™ F-150 Real-time PCR device of WizBio Solution Inc and then repeats 1) applying heat at 95° C. for 10 seconds, 2) applying heat at 60° C. for 20 seconds, and 3) applying heat at 72° C. for 10 seconds for a total of 40 times. The experimental results were analyzed using a WizDx™ Recoder.

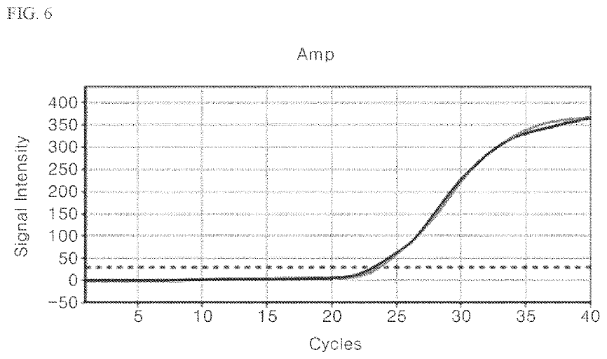

FIG. 6 is a picture showing a result of amplifying a canine parvo virus DNA sample extracted according to an embodiment of the present disclosure and a control group by real-time PCR.

The grey line in FIG. 6 shows the result of the experiment using a method for extracting nucleic acid according to the present disclosure and the black line in FIG. 6 shows the result of the experiment using the control group.

Referring to FIG. 6, it may be seen that a syringe-type nucleic acid extraction apparatus, a nucleic acid extraction kit, and a method for extracting the nucleic acid according to the present disclosure enable excellent degree of separation of virus DNA from a blood sample.

### <Experimental Example 2> Extraction of Canine Distemper Virus DNA

<2-1> Purification of Virus RNA

100 μl of blood mixed with canine distemper virus (Central Vaccine Research Institute, CDV, Rockborn, 103.5TCID50) was mixed with the lysis solution contained in well 1 of a cartridge and was left at room temperature for 5 minutes to dissolve the blood Nucleic acid was separated and purified from device nucleic acid purification filter by reciprocating a piston 5 times, reciprocating the piston once in well 2, reciprocating the piston once in well 3, reciprocating the piston once in well 4, reciprocating the piston 15 times in well 5, and finally reciprocating the piston 5 times in well 6. The finally eluted purified specimen was used as a template for a real-time RT-PCR reaction.

<2-2> RT-PCR Execution

RT-PCR was performed using virus RNA purified in Embodiment 2-1 as a template. A CDV RNA extraction method using WizPure™ Viral DNA/RNA Mini Kit (Cat# W73050-100) of WizBio Solution, Inc. was used as a control group for the RT-PCR reaction.

A forward primer 5′-AGCTAGTTTCATCTTAACTATCAAATT-3′ (SEQ ID NO: 4), a reverse primer 5′-TTAACTCTCCAGAAAACTCATGC-3′ (SEQ ID NO 5), and a probe primer 5′-FAM-ACCCAAGAGCCGGATCATATTCAATGC-BHQI-3′ (SEQ ID NO. 6) as a primer used for the real time RT-PCR were a nucleoprotein (N) gene of a canine distemper virus.

The real-time RT-PCR heats at 50° C. for 5 minutes and at 95° C. for 30 seconds using a WizDx™ F-150 Real-time PCR device of Wiz Bio Solution Inc. and then repeats 1) applying heat at 95° C. for 10 seconds, 2) applying heat at 60° C. for 20 seconds, and 3) applying heat at 72° C. for 10 seconds, for a total of 40 times. The experimental results were analyzed using a WizDx™ Recoder.

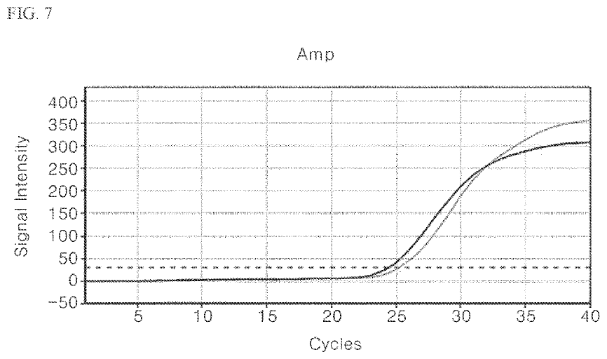

FIG. 7 is a picture showing a result of amplifying a canine distemper virus RNA sample extracted according to an embodiment of the present disclosure and a control group by Real-time RT-PCR.

The grey line in FIG. 7 shows the result of the experiment using a method for extracting nucleic acid according to the present disclosure and the black line in FIG. 7 is the result of the experiment using the control group.

Referring to FIG. 7, it may be seen that a syringe-type nucleic acid extraction apparatus, a nucleic acid extraction kit, and the nucleic acid extraction method according to the present disclosure enable excellent degree of separation of viral RNA from a blood sample.

Specific embodiments of the syringe-type nucleic acid extraction apparatus, the nucleic acid extraction kit, and the nucleic acid extraction method according to an embodiment of the present disclosure have been described hereinabove, but various modifications can be made within a limitation that does not deviate from the scope of the present disclosure.

Therefore, the scope of the present disclosure is not limited to the described embodiments and should be defined by claims described below and equivalents to the claims.

That is, it should be understood that the above-described embodiments are illustrative in all respects and not limiting, and the scope of the present disclosure is indicated by the claims described below rather than the detailed description, and the meaning, the scope of the claims, and all changes or modifications derived from the equivalents thereof are included in the scope of the present disclosure.

<Description of Sequence List>

SEQ ID NO: 1 is a nucleic acid sequence of a forward primer for Real-time PCR as a capsid protein (VP2) gene of a canine parvo virus.

SEQ ID NO: 2 is a nucleic acid sequence of a reverse primer for Real-time PCR as a capsid protein (VP2) gene of a canine parvo virus.

SEQ ID NO: 3 is a nucleic acid sequence of a probe primer for Real-time PCR as a capsid protein (VP2) gene of a canine parvo virus.

SEQ ID NO. 4 is a nucleic acid sequence of a forward primer for Real-time RT-PCR as a nucleoprotein (N) gene of a canine distemper virus.

SEQ ID NO: 5 is a nucleic acid sequence of a reverse primer for Real-time RT-PCR as a nucleoprotein (N) gene of a canine distemper virus.

SEQ ID NO 6 is a nucleic acid sequence of a probe primer for Real-time RT-PCR as a nucleoprotein (N) gene of a canine distemper virus.

### DESCRIPTION OF REFERENCE NUMERALS

10: Nucleic acid purification filter
20: Syringe
23: Cylinder
25: Piston
30. Tip
40: Coupler
43: Outer wall protrusion at lower end of syringe
45: Inner wall groove at upper end of tip
47: Nucleic acid purification filter support structure at lower side of syringe
49: Nucleic acid purification filter support structure at upper side of tip
100: Nucleic acid extraction apparatus

1. A nucleic acid extraction apparatus, comprising:
a nucleic acid purification filter;
a syringe contacting an upper surface of the nucleic acid purification filter and comprising a cylinder and a piston;
a tip having an upper surface contacting a lower surface of the nucleic acid purification filter and a lower side through which prepared specimen, washing liquid, or nucleic acid elution buffer moves; and
a coupler configured to connect a lower end of the syringe to the upper end of the tip and into which the nucleic acid purification filter is inserted.

2. The nucleic acid extraction apparatus of claim 1, wherein the coupler comprises an outer wall protrusion at a lower end of the syringe and an inner wall groove at an upper end of the tip and the outer wall protrusion and the inner wall groove may be male/female coupled.

3. The nucleic acid extraction apparatus of claim 1, wherein the coupler further comprises a structure to support the nucleic acid purification filter at a lower side of the syringe and an upper side of the tip.

4. The nucleic acid extraction apparatus of claim 3, wherein the structure to support the nucleic acid purification filter at the lower side of the syringe has a wagon wheel shape and the structure to support the nucleic acid purification filter at the upper side of the tip comprises a plurality of right-triangular protrusions from an inner wall of the upper side of the tip.

5. The nucleic acid extraction apparatus of claim 1, wherein nucleic acid purification filter comprises one selected from nanofibers, glass fibers, or silica membranes.

6. The nucleic acid extraction apparatus of claim 5, wherein the nucleic acid purification filter is surface-treated with a cationic polymer.

7. A nucleic acid extraction kit, further comprising a cartridge processing by adding the specimen, in addition to the syringe-type nucleic acid extraction apparatus according to claim 1.

8. The nucleic acid extraction kit of claim 7, wherein the cartridge comprises six wells and each well is filled with a buffer need to purify the nucleic acid or is empty for drying.

9. A method for extracting nucleic acid using the nucleic acid extraction apparatus of claim 1, comprising:
(a) preparing a specimen by dissolving the sample in a lysis solution;
(b) immersing the tip of the syringe-type nucleic acid extraction apparatus in the specimen prepared at the (a) and vertically reciprocating the piston of the syringe to adsorb nucleic acid of the prepared specimen to the nucleic acid purification filter;
(c) immersing the tip in the washing liquid and vertically reciprocating the piston of the syringe to wash the nucleic acid purification filter;
(d) vertically reciprocating the piston of the syringe to dry the nucleic acid purification filter; and
(e) immersing the tip in the nucleic acid elution buffer and vertically reciprocating the piston of the syringe to separate nucleic acid in the nucleic acid purification filter

10. The method of claim 9, wherein, at the step (a), the specimen is prepared by dissolving the sample containing the nucleic acid in a solution of a nonionic surfactant.

11. The method of claim 10, wherein the sample containing the nucleic acid is one selected from the group consisting of whole blood, plasma, serum, blood card, saliva, feces, tissue cells, and sputum.

12. The method of claim 9, wherein the washing at the (c) is performed in three steps.
